(19) United States
(12) Patent Application Publication
(10) Pub. No.: US 2012/0064013 A1
(43) Pub. Date: Mar. 15, 2012

(54) FOAMABLE TOPICAL COMPOSITION

(76) Inventors: David MARCOS, Kibbutz Maabarot (IL); Milane Haran, Kibbutz Shfayim (IL)

(21) Appl. No.: 13/226,761

(22) Filed: Sep. 7, 2011

Related U.S. Application Data
(63) Continuation-in-part of application No. PCT/IL2010/000096, filed on Feb. 3, 2010.
(60) Provisional application No. 61/382,648, filed on Sep. 14, 2010.
(60) Provisional application No. 61/382,888, filed on Sep. 14, 2010.

(51) Int. Cl. A61K 9/12; A61K 33/30; A61K 31/728; A61K 36/00; A61K 8/92; A61P 17/04; A61P 17/00; A61P 17/06; A61P 17/10; A61P 17/02; A61P 29/00; A61P 31/10; A61K 47/44; A61Q 19/00

(52) U.S. Cl. 424/43; 514/772; 424/642; 514/54; 424/725

(57) ABSTRACT

A foamable composition for topical application, the composition comprising a water in oil emulsion which is devoid of gelling agents.

This application is a Continuation-in-Part Application of, and claims priority from, PCT Application No. PCT/IL2010/000096, filed on Feb. 3, 2010, which in turn claims priority from Israeli Application No. IL 197464, which was filed on Mar. 8, 2009; and also claims priority from US Provisional Application Nos. 61/382,648 and 61/382,888; both filed on Sep. 14, 2010; all of which are hereby incorporated by reference as if fully set forth herein.

### FIELD OF THE INVENTION

The present invention relates to the field of chemical compositions, and more specifically to a foamable composition for topical application, the composition comprising a water in oil emulsion.

### BACKGROUND OF THE INVENTION

Topical application involves the absorption of a formulation by and/or through skin, mucous membrane or wound tissue. Topical applications may also be used to protect the skin or MUCOUS membrane from external irritants such as chemicals, biological fluids, sunlight, etc.

Conventional topical vehicles such as creams, lotions and ointments often have attributes that reduce patient compliance and compromise efficacy. For example ointments containing white petroleum as the carrier often form an impermeable barrier, so that metabolic products and excreta from the area to which they are applied are not easily removed or drained away. Furthermore, it may be difficult for an active agent dissolved in the carrier to pass through the white petrolatum barrier layer to the skin, so the efficacy of the drug is reduced. In addition, ointments and creams often do not create an environment for promoting normal respiration of the skin.

Foams for topical application are pressurized delivery forms containing one or more ingredients that, upon valve actuation, emit a fine dispersion of liquid and/or solid materials in a gaseous medium. Topical foams utilize compressed gases of various types as propellants, including but not limited to hydrocarbon gases (propane, butane, and isobutane), nitrogen, carbon dioxide, freons, chlorofluorocarbons, and fluorocarbons.

Foam formulations are generally easier to apply, are less dense, and spread more easily than other topical dosage forms. Foams may be formulated in various ways to provide emollient or drying functions to the skin, depending on the formulation constituents.

Use of emulsions in foam compositions is known. Emulsion systems provide a two-phase system including lipophilic or hydrophobic components in one phase and hydrophilic components in the second phase. The foamed emulsion typically is an oil-in-water emulsion in which the hydrophobic component is dispersed in the aqueous continuous phase.

For example, WO 04/037225 discloses an oil in water emulsion comprising 80-98% water; and US 20040106688 discloses an oil in water emulsion comprising from 50 to 97% by weight of a water phase.

US 20080044444 discloses foamable compositions comprising a dicarboxylic acid or ester derivative thereof in which the dicarboxylic acid or ester derivative is a stabilizing emollient and or has a therapeutic effect.

US 20060281823 discloses a foamable water-in-oil type emulsion composition comprising diglyceride-containing fats and oils and a liquid sugar, having improved in-mouth meltability and thermostability with a low specific gravity and a good eating texture for use in buttercreams or similar products. The application does not disclose the use of such a composition comprising an active pharmaceutical ingredient. Foams and, in particular, foam emulsions are complicated systems which do not form under all circumstances. Slight shifts in foam emulsion composition, such as by the addition of active ingredients, may destabilize the foam.

WO 2007/007208 discloses a water in oil foam composition comprising a plurality of surfactants, wherein a total Hydrophilic-lipophilic balance (“HLB”) for a water in oil composition is stated as being between about 2 and about 9. The formulations in this application include at least one gelling agent, which control the residence of the composition in the target site. Self-spreading of the foam is thus limited in the prior art composition. Addition of gelling agents often complicates manufacture of the product as these agents are sometimes difficult to dissolve, they may form lumps and usually greatly increase production times and costs.

### SUMMARY OF THE INVENTION

The prior art does not disclose a foamable, water in oil emulsion for topical application, which is devoid of a gelling agent and/or benzalkonium chloride.

The present invention provides a foamable composition for topical application, the composition comprising a water in oil emulsion, which is devoid of a gelling agent and/or benzalkonium chloride.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Where ranges are given, endpoints are included within the range. Furthermore, it is to be understood that unless otherwise indicated or otherwise evident from the context and understanding of one of ordinary skill in the art, values that are expressed as a range can assume any specific value or subrange within the stated range in different embodiments of the invention, to the tenth of the unit of the lower limit of the range, unless the context clearly dictates otherwise. Where a percentage is recited in reference to a value that intrinsically has units that are whole numbers, any resulting fraction may be rounded to the nearest whole number.

In case of conflict, the patent specification, including definitions, will control. In addition, the materials, methods, and examples are illustrative only and not intended to be limiting.

### DESCRIPTION OF THE PREFERRED EMBODIMENTS

The present invention provides, in at least some embodiments, a foamable composition comprising an emulsion for topical application.

According to some embodiments, the composition may comprise a water in oil emulsion. According to other embodiments of the present invention, the composition may comprise an oil in water emulsion.

In some demonstrative embodiments, the composition of the present invention may be particularly useful for treatment and/or prevention of one or more skin conditions, for example, as described in detail below.

According to some demonstrative embodiments of the present invention, the composition may include one or more components and/or properties to enable the treatment and/or prevention of one or more skin conditions, e.g., as described herein.

According to other demonstrative embodiments of the present invention, the composition described herein may be effective for the treatment and/or prevention of one or more skin conditions, e.g., as described herein, while being devoid of one or more components, for example, one or more components that may cause irritation to the skin, for example, while being devoid of a gelling agent and/or a preservative, e.g., benzalkonium chloride.

According to some embodiments, the present invention provides a foamable composition comprising a water in oil emulsion, the emulsion comprising, for example, up to about 40% (w/w) oil and up to about 60% (w/w) water.

According to some embodiments, the present invention provides a foamable composition comprising a water in oil emulsion, the emulsion comprising up to about 40% (w/w) oil, up to about 60% (w/w) water and at least one surfactant, providing, for example, a total HLB of less than about 7.

According to some embodiments, the composition may comprise an emulsion, the emulsion comprising at least one of zinc oxide, hyaluronic acid or zinc hyaluronate, optionally as an active ingredient, although the use of other active ingredients, including natural ingredients of plant and/or animal origin, such as, for example, Euphrasia Mallow, Chamomile, Hamamelis, or Aloe is considered as being within the scope of the invention.

Zinc oxide is commonly used to treat minor burns and skin irritation, and is a preferred ingredient in many creams and ointments for the treatment or prevention of various skin conditions, e.g., diaper rash.

According to some demonstrative embodiments of the present invention, the foamable composition may comprise a water in oil emulsion comprising at least one surfactant, providing a total HLB of less than about 7, wherein the composition is devoid of a gelling agent. According to some embodiments, the emulsion may further comprise at least one of zinc oxide, hyaluronic acid, zinc hyaluronate or a plant extract.

In some aspects of this embodiment, the composition may comprise up to about 25% (w/w of total composition) zinc oxide. According to some embodiments of the present invention, zinc oxide is preferably present at a concentration in the range of from about 2% to about 20% w/w of total composition, more preferably about 10% w/w of total composition.

According to some embodiments of the present invention, the composition may further comprise up to about 40% (w/w of total composition) oil as a carrier in a water in oil emulsion.

According to some demonstrative embodiments of the present invention, the oil described herein may have a certain degree of polarity.

As is known in the art, polarity is a measure of the unequal distribution of electrons across a molecule. Different level of polarity may be attributed to different molecules according to their atom content and/or structure.

Polar oils may contain heteroatoms that differ in electronegativity. This results in a dipole moment. Typical polar oils are fatty alcohols, esters and triglycerides. An example of a polar molecule is water, wherein the electrons are not evenly distributed thus creating a dipole.

Nonpolar oils may be hydrocarbons. They lack an electronegative element like oxygen, which results in their typical hydrocarbon feel. An example of a non-polar molecule is the methane molecule. In the methane molecule (CH₄) the four C—H bonds are arranged tetrahedrally around the carbon atom. Each bond has a certain polarity (though not very strong in this case). However, the bonds are arranged symmetrically so there is no overall dipole in the molecule.

The degree of polarization of a bond may be measured in percent ionic character, and one can determine this value from the difference in electronegativity of two atoms using various methods (El-Mahrab-Robert et al., Assessment of oil polarity: Comparison of evaluation methods, International Journal of Pharmaceutics 348 (2008) 89-94). For example, water contains the bond O—H between oxygen and hydrogen. The electronegativity of O is 3.5 and H is 2.1, so they differ by 1.4. On a chart of ionic character, one may find this bond is 39 percent ionic, thus a compound containing O—H will be quite polar. The ionic character of a carbon-hydrogen bond is only 4 percent, so a compound containing only C—H would be nonpolar.

According to some embodiments, some oils described herein may contain few charged molecules, and may be referred to herein as having low polarity. According to other embodiments, some oils described herein may contain charged molecules and may be referred to herein as having medium and/or high polarity. In some demonstrative embodiments, the oil of the present invention may have a low polarity and may include, for example, one or more of mineral oil, triglyceride oil, squalane, tocopherol or its derivatives, avocado oil, macadamia oil, corn oil, olive oil, sesame oil, peanut oil, octyl dodecanate, or oleic acid decyl ester, or combinations thereof.

Alternatively or additionally, the oil may have a medium to high polarity. Non-limiting examples of suitable oils may include isopropyl myristate, isopropyl palmitate, caprylic/capric triglycerides, propylene glycol dicaprylate/dicaprate, decyl oleate, dibutyl adipate, or hexyl laurate, or combinations thereof.

According to some demonstrative embodiments, the oil may include a combination of a low polarity oil and a medium to high polarity oil.

According to a preferred embodiment, the oil of the present invention comprises isopropyl myristate and mineral oil. According to some aspects of this embodiment, the mineral oil is optionally and preferably present at a concentration of from about 20 to about 40% w/w of total composition, and said isopropyl myristate is present at a concentration of from about 5 to about 15% and more preferably from about 6 to about 10%, w/w of total composition.

According to some embodiments of the present invention, the composition may comprise at least one surfactant, the surfactant or combination of surfactants may provide a total HLB of less than about 7. Optionally and preferably, at least one surfactant may comprise a fatty acid ester having an HLB of less than about 7.

Examples of suitable fatty acid esters may include but are not limited to sorbitan fatty acid esters (such as sorbitan monolaurate; sorbitan mono palmitate; sorbitan monooleate, sorbitan dioleate; sorbitan trioleale; sorbitan sesquioleate; sorbitan monolaurate; sorbitan monoisostearate; sorbitan diisostearate; sorbitan sesquistearate); sucrose fatty acid esters (such as sucrose monopalmitate; sucrose monostearate; sucrose distearate; sucrose polystearate); propylene glycol fatty acid esters (such as propylene glycol stearate; propylene glycol alginate); glyceryl fatty acid esters (such as glyceryl monooleate; glyceryl monostearate; glyceryl myristate).

Optionally and preferably, the fatty acid ester surfactant may comprise glyceryl monooleate. More preferably, the glyceryl monooleate is present at a concentration in the range of from about 0.1% to about 7% w/w of total composition, and most preferably, in the range of from about 0.1 to about 3.5% w/w of total composition.

Additionally or alternatively, the fatty acid ester surfactant may comprise sucrose polystearate. Optionally and preferably, sucrose polystearate is present at a concentration of up to about 6% w/w of total composition. Optionally and more preferably, sucrose polystearate is present at a concentration of from about 0.3% to about 2% w/w of total composition.

Additionally or alternatively, the fatty acid ester surfactant may comprise sucrose polystearate and sucrose distearate. Optionally and preferably, sucrose polystearate is present at a concentration of up to about 3% w/w of total composition, and sucrose distearate is present at a concentration of up to about 1% w/w of total composition. Optionally and more preferably, sucrose polystearate is present at a concentration of about 0.3% w/w of total composition, and sucrose distearate is present at a concentration of about 0.1% w/w of total composition.

Also additionally or alternatively, the fatty acid surfactant may comprise sucrose distearate. Optionally and preferably, sucrose distearate is present at a concentration of up to about 4% w/w of total composition. Optionally and more preferably, sucrose distearate is present at a concentration of from about 0.3% to about 1% w/w of total composition.

According to some embodiments of the present invention, the composition may further comprise a co-emulsifier, such as, for example, beeswax, lanolin, cetyl alcohol, stearyl alcohol, cetostearyl alcohol, stearic acid, or palmitic acid. Optionally and preferably, the beeswax may be present at a concentration of up to about 6% w/w of total composition. Optionally and more preferably, the beeswax is present at a composition of from about 2% to about 5% w/w of total composition. Also optionally and preferably, lanolin is present at a concentration of up to about 7% w/w of total composition. Optionally and more preferably, the lanolin is present at a concentration of from about 2% to about 5% w/w of total composition.

According to some embodiments, the composition of the present invention may further comprise panthenol. According to some embodiments, Panthenol may act as a humectant, emollient and/or moisturizer, for example, to improve hydration, reduce itching and/or inflammation of the skin, accelerate and/or improve healing of wounds, e.g., epidermal wounds. Optionally and preferably, panthenol is present at a concentration of up to about 5% w/w of total composition. Optionally and more preferably, panthenol is present at a concentration of about 2% w/w of total composition.

According to some embodiments, the composition of the present invention may further comprise aloe vera extract, for example, in an amount effective in the treatment of wounds and/or skin irritations. Optionally and preferably, the aloe vera extract is present at a concentration of up to about 1% w/w of total composition. Optionally and more preferably, the aloe vera extract is present at a concentration of about 0.1% w/w of total composition.

According to some embodiments, the composition of the present invention may further comprise a preservative (such as, for example, benzalkonium chloride, sodium benzoate, benzoic acid, benzyl alcohol, a paraben or salt thereof, imidurea, potassium sorbate, sorbic acid, phenoxyethanol, chlorocresol, or bronopol, or mixtures thereof).

In some embodiments, especially according to the embodiments wherein the composition is devoid of benzalkonium chloride, a paraben may be used, for example, to act as a preservative. According to some embodiments, the paraben may be selected from the group consisting of methyl paraben, methyl paraben sodium, propyl paraben, propyl paraben sodium, or mixtures thereof.

Optionally and preferably, at least one of methyl paraben and methyl paraben sodium is present at a concentration of up to about 0.2%, and more preferably at a concentration of about 0.18% w/w of total composition.

Optionally and preferably, at least one of propyl paraben and propyl paraben sodium is present at a concentration of up to about 0.1%, and more preferably at a concentration of about 0.02% w/w of total composition.

According to some embodiments of the present invention, a preservative such as benzalkonium chloride may be present in the composition, e.g., at a concentration of up to about 1% w/w of total composition. In some embodiments, the preservative may be present at a concentration of about 0.2% w/w of total composition.

Benzalkonium chloride, also known as alkyldimethylbenzylammonium chloride (ADBAC) is a mixture of alkylbenzyldimethylammonium chlorides of various even-numbered-alkyl chain lengths. This product is a nitrogenous cationic surface-acting agent belonging to the quaternary ammonium group. Standard concentrates are manufactured as 50% and 80% w/w solutions, and sold under trade names such as BC50, BC80, BAC50, BAC80, etc. Benzalkonium chloride solutions of 10% or more are toxic to humans, causing irritation to the skin and mucosa. The present inventors have found that concentrations of less than 10%, when used as preservative in foam compositions, may have irritant effects on human skin, particularly in combination with certain other excipients.

According to other embodiments of the present invention, the composition may comprise at least one surfactant and may be devoid of benzalkonium chloride.

According to some embodiments, the present invention may provide a foamable composition comprising an emulsion, said emulsion comprising at least one surfactant and being devoid of benzalkonium chloride, for use in treating a skin condition.

According to some embodiments, the composition of the present invention may further comprise a surfactant selected from the group consisting of self emulsifying surfactants such as glyceryl stearate (with) PEG-100 stearate (e.g. Arlacel 170®, Simulsol 165®), sorbitan stearate (with) sucrose cocoate (e.g. Arlatone 2121®), PEG-30 dipolyhydroxystearate (e.g. Arlacel P135®), and ceteth-2 (e.g. Brij 52®), or mixtures thereof.

Optionally and preferably, at least one of glyceryl stearate (with) PEG-100 stearate and sorbitant stearate (with) sucrose cocoate may be present at a concentration of up to about 1%, and more preferably about 0.2% w/w of total composition.

Optionally and preferably, PEG-30 dipolyhydroxystearate may be present at a concentration of up to about 1%, and more preferably about 0.4% w/w of total composition.

Optionally and preferably, ceteth-2 may be present at a concentration of up to about 5%, and more preferably about 3% w/w of total composition.

According to some embodiments, the composition of the present invention may further comprise Bisabolol, preferably at a concentration of up to about 1% w/w of the total composition and more preferably, at a concentration of about 0.2% w/w of the total composition. According to some embodiments, Bisabolol, a derivative of M. chamomilla (German chamomile), may act as a penetration enhancer, exhibiting, for example, anti-inflammatory, anti-irritant, anti-bacterial, and/or non-allergenic properties.

According to some embodiments, the composition of the present invention may further comprise at least one of a chelating agent, such as EDTA as well as its sodium or calcium salts; a solvent, for example a hydrophilic liquid, such as propylene glycol or glycerin; an emulsion stabilizer, such as magnesium sulfate, or combinations thereof.

Optionally and preferably, the chelating agent may comprise disodium EDTA, more preferably at a concentration of up to about 1% w/w of total composition, most preferably at a concentration of about 0.3% w/w of total composition.

Optionally and preferably, the hydrophilic liquid may comprise propylene glycol, more preferably at a concentration of from about 1% to about 3% w/w of total composition, most preferably at a concentration of about 2% w/w of total composition.

Optionally and preferably, the emulsion stabilizer may comprise magnesium sulfate, more preferably at a concentration of up to about 1% w/w of total composition, most preferably at a concentration of about 0.2% w/w of total composition.

According to some embodiments, the emulsion stabilizer may comprise xanthan gum, more preferably at a concentration of up to about 1% w/w of total composition, most preferably at a concentration of about 0.25% w/w of total composition.

According to some embodiments, the foamable composition of the present invention may be adapted for delivery from a pressurized container, for example, wherein a foam may be formed upon expulsion from the container.

According to some embodiments of the present invention, the composition may further comprise at least one propellant, such as, for example, one or more hydrocarbon gases (propane, butane and isobutane) and/or fluorocarbons (such as Dymel 134a®).

The composition of the present invention may be useful, in some embodiments, for the treatment and/or prevention of a skin condition, such as, for example, diaper rash, psoriasis, eczema, urticaria, scabies, acne, alopecia areata, bullous pemphigoid, dermatitis (including contact dermatitis, atopic dermatitis, dermatitis herpetiformis, seborrheic dermatitis, nummular dermatitis, stasis dermatitis, and perioral dermatitis), eczema, urticaria, scabies, acne, alopecia areata, bullous pemphigoid, impetigo, keloids, pruritis, rosacea, vitiligo, burns, irritation, inflammation, fungal infection, dry skin, skin allergies, diabetic skin conditions, decubitus ulcers and the like.

According to some demonstrative embodiments of the present invention, the composition may be useful for the treatment and/or prevention of one or more skin conditions which may occur and/or develop due to one or more of rubbing, chafing, abrasion and the like. For example, the composition may be used by people and/or patients which may be more prone to develop one or more of the above mentioned skin conditions, such as, athletes, e.g., bicycle riders and runners, soldiers, obese people, and the like. According to some embodiments, a person may be considered as obese when the Body mass index (BMI) greater than 30 kg/m².

The composition of the present invention may be useful, in some embodiments, for providing a skin soothing and anti-itching effect.

The composition of the present invention may preferably be useful for treatment and/or prevention of skin conditions such as dermatitis, including diaper dermatitis (diaper rash), both in infants and in incontinent adults.

As used herein, the term “treating” may include abrogating, substantially inhibiting, slowing and/or reversing the progression of a condition, substantially ameliorating clinical and/or aesthetical symptoms of a condition and/or substantially preventing and/or diminishing the appearance of clinical and/or aesthetical symptoms of a condition.

Diaper rash is a generalized term indicating any skin irritation, regardless of cause, that develops in the diaper-covered region. While diaper rash is generally thought to affect infants and toddlers, any individual wearing a diaper (for example, an incontinent adult) is a candidate to develop this condition. Contact dermatitis is the most common category of diaper rash Skin involvement may vary from mild redness to erosion of the top layers of skin Other categories include skin infections and allergic reactions. It is very important that no excipient which may result in irritation of the skin be present in a composition for treatment of diaper rash, especially when the formulation is used on the highly sensitive skin of an infant or toddler.

According to some embodiments, the foam formulations and/or compositions of the present invention, may be particularly useful for treatment and/or prevention of diaper rash in incontinent adult patients, such as geriatric patients, or physically or mentally handicapped adults. For such patients, who require the use of diapers, a formulation for prevention of diaper rash must often be administered by a single carer, using one hand, while raising and holding the posterior region of the patient with the other hand. Foam formulations may be significantly easier to apply in such circumstances than other dosage forms which must be applied and/or spread by hand. Furthermore, application of a foam composition does not require the hand of the carer to contact the skin of the patient, hence the hand does not have to be carefully cleaned, or a sterile glove changed, immediately after use. Additionally, a patient suffering from soreness due to diaper rash experiences less discomfort due to contact with the hand of a carer during application of the composition. In addition, use of a composition as described herein in accordance with some demonstrative embodiments, may prevent or diminish embarrassment to the patient and care giver who is required to apply the product to intimate parts of the patient.

Oil in water foam compositions may be problematic for treatment of diaper rash, for example, since the composition may be easily washed away by the urine and/or fecal excretions of a subject. Water in oil compositions are preferable in this respect; however, stable water in oil foaming compositions are more difficult to formulate.

The composition of the present invention may preferably be dispensed from a pressurized container in an amount which provides a suitable volume for containment within a diaper, without oozing outwards. According to some embodiments. The absence of a gelling agent in the foam composition of the present invention may preferably provide a foam which is less stable than conventional foams, and which is not limited to residence at the application site, enabling, for example, self-spreading of the foam upon application from a pressurized container.

According to some embodiments, the composition of the present invention may be a cosmetic composition for topical application. Such a composition may be useful for improving the appearance and/or texture of the skin.

According to some embodiments, the composition of the present invention may be provided as a medical device.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As used herein the term “about” refers to ±10%.

Pharmaceutical compositions suitable for use in the context of the present invention include compositions wherein the active ingredient is contained in an amount effective to achieve the intended purpose. More specifically, a “therapeutically effective amount” means an amount of active ingredient effective to prevent, alleviate, or ameliorate a skin and/or mucous membrane condition.

Determination of a therapeutically effective amount is well within the capability of those skilled in the art, especially in light of the detailed disclosure provided herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Example 1

| Material | Amount (% w/w) |
|---|---|
| Light mineral oil | 28.8 |
| Isopropyl myristate | 7 |
| Lanolin | 5 |
| White beeswax | 3 |
| Sucrose distearate | 0.3 |
| Sucrose polystearate | 0.3 |
| Glyceryl monooleate | 2 |
| Zinc oxide | 10 |
| Dexpanthenol | 2 |
| Methyl paraben sodium | 0.18 |
| Propyl paraben sodium | 0.02 |
| Aloe vera dry extract | 0.1 |
| Purified water | 40.9 |

### Example 2

| Material | Amount (% w/w) |
|---|---|
| Light mineral oil | 24 |
| Isopropyl myristate | 6 |
| Lanolin | 2 |
| White beeswax | 4 |
| Sucrose distearate | 1 |
| Sucrose polystearate | 1 |
| Glyceryl monooleate | 3 |
| Zinc oxide | 10 |
| Dexpanthenol | 2 |
| Methyl paraben sodium | 0.18 |
| Propyl paraben sodium | 0.02 |
| Aloe vera dry extract | 0.1 |
| Purified water | 46.7 |

### Example 3

| Material | Amount (% w/w) |
|---|---|
| Light mineral oil | 32.8 |
| Isopropyl myristate | 6 |
| Lanolin | 2 |
| White beeswax | 5 |
| Sucrose distearate | 0.5 |
| Sucrose polystearate | 0.5 |
| Glyceryl monooleate | 2 |
| Zinc oxide | 10 |
| Dexpanthenol | 2 |
| Methyl paraben | 0.18 |
| sodium | |
| Propyl paraben sodium | 0.02 |
| Aloe vera dry extract | 0.1 |
| Purified water | 45 |

### Example 4

| Material | Amount (% w/w) |
|---|---|
| Light mineral oil | 39.8 |
| Isopropyl myristate | 6 |
| Lanolin | 2 |
| White beeswax | 5 |
| Sucrose distearate | 0.5 |
| Sucrose polystearate | 0.5 |
| Glyceryl monooleate | 3.5 |
| Zinc oxide | 10 |
| Dexpanthenol | 2 |
| Methyl paraben | 0.18 |
| sodium | |
| Propyl paraben sodium | 0.02 |
| Aloe vera dry extract | 0.1 |
| Sodium chloride | 0.5 |
| Purified water | 30 |

### Example 5

| Material | Amount (% w/w) |
|---|---|
| Light mineral oil | 23 |
| Isopropyl myristate | 6 |
| Lanolin | 2 |
| White beeswax | 2 |
| Sucrose distearate | 0.3 |
| Sucrose polystearate | 1 |
| Glyceryl monooleate | 1.2 |
| Zinc oxide | 10 |
| Dexpanthenol | 2 |
| Methyl paraben | 0.18 |
| sodium | |
| Propyl paraben sodium | 0.02 |
| Aloe vera dry extract | 0.1 |
| Purified water | 52.2 |

### Example 6

| Material | Amount (% w/w) |
|---|---|
| Light mineral oil | 26.6 |
| Isopropyl myristate | 6 |
| Lanolin | 2 |
| White beeswax | 3.5 |
| Sucrose distearate | 0.3 |
| Sucrose polystearate | 1 |
| Glyceryl monooleate | 1.5 |
| Zinc oxide | 10 |
| Dexpanthenol | 2 |
| Methyl paraben | 0.18 |
| sodium | |
| Propyl paraben sodium | 0.02 |
| Aloe vera dry extract | 0.1 |
| Purified water | 46.8 |

### Example 7

| Material | Amount (% w/w) |
|---|---|
| Light mineral oil | 23 |
| Isopropyl myristate | 6 |
| Lanolin | 2 |
| White beeswax | 3 |
| Sucrose distearate | 0.3 |
| Sucrose polystearate | 1 |
| Glyceryl monooleate | 2 |
| Zinc oxide | 10 |
| Dexpanthenol | 2 |
| Methyl paraben | 0.18 |
| sodium | |
| Propyl paraben sodium | 0.02 |
| Aloe vera dry extract | 0.1 |
| Purified water | 50.4 |

### Example 8

| Material | Amount (% w/w) |
|---|---|
| Light mineral oil | 25 |
| Isopropyl myristate | 6 |
| Lanolin | 2 |
| White beeswax | 3 |
| Sucrose distearate | 0.3 |
| Sucrose polystearate | 1 |
| Glyceryl monooleate | 2 |
| Zinc oxide | 10 |
| Dexpanthenol | 2 |
| Methyl paraben | 0.18 |
| sodium | |
| Propyl paraben sodium | 0.02 |
| Aloe vera dry extract | 0.1 |
| Purified water | 48.4 |

### Example 9

| Material | Amount (% w/w) |
|---|---|
| Light mineral oil | 23 |
| Isopropyl myristate | 6 |
| Lanolin | 2 |
| White beeswax | 2 |
| Sucrose distearate | 0.3 |
| Sucrose polystearate | 1 |
| Glyceryl monooleate | 1.2 |
| Zinc oxide | 10 |
| Dexpanthenol | 2 |
| Methyl paraben | 0.18 |
| sodium | |
| Propyl paraben sodium | 0.02 |
| Aloe vera dry extract | 0.1 |
| Purified water | 52.2 |

### Example 10

| Material | Amount (% w/w) |
|---|---|
| Light mineral oil | 23 |
| Isopropyl myristate | 6 |
| Lanolin | 2 |
| White beeswax | 2 |
| Sucrose distearate | 1 |
| Sucrose polystearate | 1.2 |
| Glyceryl monooleate | 0.12 |
| Zinc oxide | 10 |
| Dexpanthenol | 2 |
| Methyl paraben | 0.18 |
| Propyl paraben | 0.02 |
| Aloe vera dry extract | 0.1 |
| Purified water | 52.38 |

### Example 11

| Material | Amount (% w/w) |
|---|---|
| Light mineral oil | 20 |
| Isopropyl myristate | 10 |
| Lanolin | 5 |
| White beeswax | 4 |
| Simulsol 165 ® | 0.2 |
| Glyceryl monooleate | 1.3 |
| Sucrose polystearate | 2 |
| Arlatone 2121 ® | 0.2 |
| Arlacel 135 ® | 0.4 |
| Brij 52 ® | 3 |
| Bisabolol | 0.2 |
| Zinc oxide | 10 |
| Disodium EDTA | 0.3 |
| Dexpanthenol | 2 |
| Propylene glycol | 2 |
| Benzalkonium chloride | 0.2 |
| (50%) | |
| Aloe vera dry extract | 0.1 |
| Magnesium sulphate | 0.2 |
| Fragrance | 0.2 |
| Purified water | 38.3 |

### Example 12

| Material | Amount (% w/w) |
|---|---|
| Light mineral oil | 26.55 |
| Isopropyl myristate | 6 |
| Lanolin | 5 |
| Beeswax substitute | 2 |
| Sucrose distearate | 0.3 |
| Glyceryl monooleate | 1.2 |
| Sucrose polystearate | 1.5 |
| Zinc oxide | 10 |
| Xanthan gum | 0.25 |
| Dexpanthenol | 2 |
| Propylene glycol | 2 |
| Methylparaben | 0.18 |
| Propylparaben sodium | 0.02 |
| Aloe vera dry extract | 0.1 |
| Magnesium sulfate | 0.2 |
| Purified water | 42 |

### Example 13

| Material | Amount (% w/w) |
|---|---|
| Light mineral oil | 25.8 |
| Isopropyl myristate | 6 |
| Lanolin | 5 |
| Beeswax substitute | 2 |
| Sucrose distearate | 0.3 |
| Glyceryl monooleate | 1.2 |
| Sucrose polystearate | 1.5 |
| Zinc oxide | 10 |
| Xanthan gum | 0.25 |
| Dexpanthenol | 2 |
| Propylene glycol | 2 |
| Phenoxyethanol | 0.7 |
| Potassium sorbate | 0.2 |
| Aloe vera dry extract | 0.1 |
| Magnesium sulfate | 0.2 |
| Purified water | 42 |

### Example 14

| Material | Amount (% w/w) |
|---|---|
| Light mineral oil | 26.7 |
| Isopropyl myristate | 6 |
| Lanolin | 5 |
| White beeswax | 2 |
| Arlacel 165 ® | 0.15 |
| Glyceryl monooleate | 1.2 |
| Sucrose polystearate | 1.5 |
| Zinc oxide | 10 |
| Xanthan gum | 0.25 |
| Dexpanthenol | 2 |
| Methylparaben sodium | 0.18 |
| Propylparaben sodium | 0.02 |
| Aloe vera dry extract | 0.1 |
| Purified water | 44.7 |

### Example 15

| Material | Amount (g) |
|---|---|
| Light mineral oil | 26.9 |
| Isopropyl myristate | 6 |
| Lanolin | 5 |
| White beeswax | 2 |
| Sucrose distearate | 0.3 |
| Glyceryl monooleate | 2 |
| Sucrose polystearate | 0.28 |
| Zinc oxide | 10 |
| Xanthan gum | 0.25 |
| Dexpanthenol | 2 |
| Methylparaben sodium | 0.18 |
| Propylparaben sodium | 0.02 |
| Aloe vera dry extract | 0.1 |
| Purified water | 44.9 |

### Example 16

| Material | Amount (g) |
|---|---|
| Light mineral oil | 383.6 |
| Isopropyl myristate | 140 |
| Lanolin | 70 |
| White beeswax | 42 |
| Simulsol 165 ™ | 4.9 |
| Glyceryl monooleate | 14 |
| Sucrose polystearate | 28 |
| Brij 52 ® | 21 |
| Zinc oxide | 140 |
| Xanthan gum | 3.5 |
| Dexpanthenol | 28 |
| Propylene glycol | 28 |
| Benzalkonium chloride | 2.8 |
| 50% | |
| Aloe vera dry extract | 1.4 |
| Magnesium sulphate | 2.8 |
| Purified water | 490 |

### Example 17

| Material | Amount (g) |
|---|---|
| Light mineral oil | 383.6 |
| Isopropyl myristate | 140 |
| Lanolin | 70 |
| White beeswax | 42 |
| Simulsol 165 ™ | 4.9 |
| Glyceryl monooleate | 28 |
| Sucrose polystearate | 14 |
| Zinc oxide | 140 |
| Xanthan gum | 3.5 |
| Dexpanthenol | 28 |
| Propylene glycol | 28 |
| Benzalkonium chloride | 2.8 |
| 50% | |
| Aloe vera dry extract | 1.4 |
| Magnesium sulphate | 2.8 |
| Purified water | 511 |

### Example 18

| Material | Amount (g) |
|---|---|
| Light mineral oil | 373.8 |
| Isopropyl myristate | 140 |
| Lanolin | 70 |
| White beeswax | 28 |
| Simulsol 165 ™ | 4.9 |
| Glyceryl monooleate | 28 |
| Sucrose polystearate | 16.8 |
| Zinc oxide | 140 |
| Xanthan gum | 3.5 |
| Dexpanthenol | 28 |
| Propylene glycol | 28 |
| Benzalkonium chloride | 2.8 |
| 50% | |
| Aloe vera dry extract | 1.4 |
| Magnesium sulphate | 2.8 |
| Purified water | 532 |

### Example 19

| Material | Amount (g) |
|---|---|
| Light mineral oil | 373.8 |
| Isopropyl myristate | 84 |
| Lanolin | 70 |
| White beeswax | 28 |
| Simulsol 165 ™ | 4.9 |
| Glyceryl monooleate | 28 |
| Sucrose polystearate | 16.8 |
| Zinc oxide | 140 |
| Xanthan gum | 3.5 |
| Dexpanthenol | 28 |
| Propylene glycol | 28 |
| Benzalkonium chloride | 2.9 |
| 50% | |
| Aloe vera dry extract | 1.4 |
| Magnesium sulphate | 2.8 |
| Purified water | 588 |

### Example 20

| Material | Amount (g) |
|---|---|
| Light mineral oil | 336 |
| Isopropyl myristate | 84 |
| Lanolin | 56 |
| Beeswax substitute | 28 |
| Simulsol 165 ™ | 2.1 |
| Glyceryl monooleate | 16.8 |
| Sucrose polystearate | 21 |
| Zinc oxide | 140 |
| Xanthan gum | 3.5 |
| Dexpanthenol | 28 |
| Propylene glycol | 28 |
| Benzalkonium chloride | 2.8 |
| 50% | |
| Aloe vera dry extract | 1.4 |
| Magnesium sulphate | 2.8 |
| Purified water | 649.6 |

### Example 21

| Material | Amount (g) |
|---|---|
| Light mineral oil | 371.7 |
| Isopropyl myristate | 84 |
| Lanolin | 70 |
| Beeswax substitute | 28 |
| Sucrose distearate | 4.2 |
| Glyceryl monooleate | 16.8 |
| Sucrose polystearate | 21 |
| Zinc oxide | 140 |
| Xanthan gum | 3.5 |
| Dexpanthenol | 28 |
| Propylene glycol | 28 |
| Methylparaben | 2.52 |
| Propylparaben sodium | 0.28 |
| Aloe vera dry extract | 1.4 |
| Magnesium sulphate | 2.8 |
| Purified water | 597.8 |

### Example 22

| Material | Amount (g) |
|---|---|
| Light mineral oil | 361.9 |
| Isopropyl myristate | 84 |
| Lanolin | 70 |
| Beeswax substitute | 28 |
| Sucrose distearate | 4.2 |
| Glyceryl monooleate | 16.8 |
| Sucrose polystearate | 21 |
| Zinc oxide | 140 |
| Xanthan gum | 3.5 |
| Dexpanthenol | 28 |
| Propylene glycol | 28 |
| Phenoxyethanol | 10.14 |
| Potassium sorbate | 2.8 |
| Aloe vera dry extract | 1.4 |
| Magnesium sulphate | 2.8 |
| Purified water | 597.8 |

### Example 23

| Material | Amount (g) |
|---|---|
| Light mineral oil | 373.8 |
| Isopropyl myristate | 84 |
| Lanolin | 70 |
| White beeswax | 28 |
| Arlacel 165 ® | 2.1 |
| Glyceryl monooleate | 16.8 |
| Zinc oxide | 140 |
| Xanthan gum | 3.5 |
| Dexpanthenol | 28 |
| Benzalkonium chloride | 2.8 |
| 50% | |
| Aloe vera dry extract | 1.4 |
| Magnesium sulphate | 2.8 |
| Purified water | 625.8 |

### Example 24

| Material | Amount (g) |
|---|---|
| Light mineral oil | 371.7 |
| Isopropyl myristate | 84 |
| Lanolin | 70 |
| White beeswax | 28 |
| Sucrose distearate | 4.2 |
| Glyceryl monooleate | 16.8 |
| Zinc oxide | 140 |
| Xanthan gum | 3.5 |
| Dexpanthenol | 28 |
| Benzalkonium chloride | 2.8 |
| 50% | |
| Aloe vera dry extract | 1.4 |
| Magnesium sulphate | 2.8 |
| Purified water | 625.8 |

### Example 25

| Material | Amount (g) |
|---|---|
| Light mineral oil | 371.7 |
| Isopropyl myristate | 84 |
| Lanolin | 70 |
| White beeswax | 28 |
| Sucrose distearate | 4.2 |
| Glyceryl monooleate | 16.8 |
| Sucrose polystearate | 21 |
| Zinc oxide | 140 |
| Xanthan gum | 3.5 |
| Dexpanthenol | 28 |
| Benzalkonium chloride | 2.8 |
| 50% | |
| Aloe vera dry extract | 1.4 |
| Purified water | 625.8 |

### Example 26

| Material | Amount (g) |
|---|---|
| Light mineral oil | 373.8 |
| Isopropyl myristate | 84 |
| Lanolin | 70 |
| White beeswax | 28 |
| Arlacel 165 ® | 2.1 |
| Glyceryl monooleate | 16.8 |
| Sucrose polystearate | 21 |
| Zinc oxide | 140 |
| Xanthan gum | 3.5 |
| Dexpanthenol | 28 |
| Methylparaben sodium | 2.52 |
| Propylparaben sodium | 0.28 |
| Aloe vera dry extract | 1.4 |
| Purified water | 625.8 |

### Example 27

| Material | Amount (g) |
|---|---|
| Light mineral oil | 377.3 |
| Isopropyl myristate | 84 |
| Lanolin | 70 |
| White beeswax | 28 |
| Sucrose distearate | 4.2 |
| Glyceryl monooleate | 28 |
| Sucrose polystearate | 4.2 |
| Zinc oxide | 140 |
| Xanthan gum | 3.5 |
| Dexpanthenol | 28 |
| Methylparaben sodium | 2.52 |
| Propylparaben sodium | 0.28 |
| Aloe vera dry extract | 1.4 |
| Purified water | 628.6 |

### Example 28

| Material | Amount (g) |
|---|---|
| Light mineral oil | 322 |
| Isopropyl myristate | 84 |
| Lanolin | 28 |
| White beeswax | 42 |
| Sucrose distearate | 4.2 |
| Glyceryl monooleate | 28 |
| Sucrose polystearate | 14 |
| Zinc oxide | 140 |
| Dexpanthenol | 28 |
| Methylparaben sodium | 2.52 |
| Propylparaben sodium | 0.28 |
| Aloe vera dry extract | 1.4 |
| Purified water | 705.6 |

### Example 29

| Material | Amount (g) |
|---|---|
| Light mineral oil | 294 |
| Isopropyl myristate | 70 |
| Lanolin | 70 |
| White beeswax | 28 |
| Sucrose distearate | 4.2 |
| Glyceryl monooleate | 16.8 |
| Sucrose polystearate | 14 |
| Zinc oxide | 140 |
| Dexpanthenol | 28 |
| Methylparaben sodium | 2.52 |
| Propylparaben sodium | 0.28 |
| Aloe vera dry extract | 1.4 |
| Purified water | 730.8 |

### Example 30

| Material | Amount (g) |
|---|---|
| Light mineral oil | 322 |
| Isopropyl myristate | 84 |
| Lanolin | 28 |
| White beeswax | 28 |
| Sucrose distearate | 14 |
| Sucrose polystearate | 1.68 |
| Glyceryl monooleate | 16.8 |
| Dexpanthenol | 28 |
| Methyl paraben sodium | 2.52 |
| Propyl paraben sodium | 0.28 |
| Aloe vera dry extract | 1.4 |
| Purified water | 873.32 |

### Example 31

| Material | Amount (g) |
|---|---|
| Light mineral oil | 345 |
| Isopropyl myristate | 90 |
| Zinc oxide | 150 |
| Lanolin | 30 |
| White beeswax | 30 |
| Sucrose distearate | 15 |
| Sucrose polystearate | 1.8 |
| Glyceryl monooleate | 18 |
| Dexpanthenol | 30 |
| Phenoxyethanol | 10.5 |
| Aloe vera dry extract | 1.5 |
| Purified water | 778.2 |

### Example 32

### Treatment and/or Prevention of Skin Conditions in Athletes

The reduction and/or prevention of skin conditions and/or irritations due to the use of one or more of the compositions described hereinabove is assessed in a clinical trial. In the trial a first group of 50 athletes (“the first group”) applies a suitable amount of a composition described hereinabove prior to conducting physical exercise, such as riding a bicycle, for 3 hours. A second group of 50 athletes (“the second group”) applies a placebo composition prior to conducting a similar physical exercise for 3 hours.

The efficiency is based on preventing and/or diminishing one or more of the following skin conditions: skin irritations, eczema, urticaria, burns, irritation, inflammation, fungal infection, dry skin and skin allergies.

The assessments for efficiency are ascertained by comparing the post-exercise skin condition of athletes from the first group to the post-exercise skin condition of athletes from the second group.

Results of the clinical study demonstrate that 12 athletes from the first group develop a skin condition in comparison to 29 athletes from the second group which develop a skin condition.

### Example 33

### Treatment and/or Prevention of Skin Condition in Obese

The reduction and/or prevention of skin conditions and/or irritations due to the use of one or more of the compositions described hereinabove is assessed in a clinical trial. In the trial a first group of 34 obese people (“the first group”) applies a suitable amount of a composition described hereinabove onto different parts of the body prone to develop skin condition, for example, skin folds and the like, three times a day for two weeks. A second group of 35 obese people (“the second group”) applies a placebo composition onto different parts of the body prone to develop skin condition, three times a day for two weeks.

The efficiency is based on preventing and/or diminishing one or more of the following skin conditions: skin irritations, eczema, urticaria, burns, irritation, inflammation, fungal infection, dry skin and skin allergies.

The assessments for efficiency are ascertained by comparing the skin condition of obese from the first group to the skin condition of obese from the second group.

Results of the clinical study demonstrate that 3 obese from the first group developed a skin condition in comparison to 24 obese from the second group which developed a skin condition.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A foamable composition comprising a water in oil emulsion, said emulsion comprising up to about 40% (w/w) oil, up to about 60% (w/w) water or hydrophilic liquid or mixture thereof, and at least one surfactant providing a total HLB value of less than about 7, wherein the composition is devoid of a gelling agent.

2. A foamable composition comprising a water in oil to emulsion, said emulsion comprising up to about 40% (w/w) oil, up to about 60% (w/w) water or hydrophilic liquid or mixture thereof, and at least one surfactant providing a total HLB value of less than about 7, wherein the composition is devoid of benzalkonium chloride.

3. The foamable composition of claim 1, comprising at least one of zinc oxide, hyaluronic acid, zinc hyaluronate, or a plant extract.

4. The foamable composition of claim 3, wherein said at least one of zinc oxide, hyaluronic acid, zinc hyaluronate, or a plant extract is present as an active ingredient.

5. The foamable composition of claim 4, comprising up to about 25% (w/w of total composition) zinc oxide.

6. The foamable composition of claim 5, wherein said surfactant comprises a fatty acid ester.

7. The foamable composition of claim 6, wherein said oil comprises one or more of a low polarity oil, or a medium to high polarity oil, or combinations thereof.

8. The foamable composition of claim 7, wherein said low polarity oil comprises one or more of mineral oil, triglyceride oil, squalane, tocopherol or its derivatives, avocado oil, macadamia oil, corn oil, olive oil, sesame oil, or peanut oil, or combinations thereof.

9. The foamable composition of claim 7, wherein said medium to high polarity oil comprises one or more of isopropyl myristate, isopropyl palmitate, caprylic/capric triglycerides, propylene glycol dicaprylate/dicaprate, decyl oleate, dibutyl adipate, or hexyl laurate, or combinations to thereof.

10. The foamable composition of claim 7, wherein said oil comprises isopropyl myristate and mineral oil.

11. The foamable composition of claim 8, wherein said mineral oil is present at a concentration of from about 20 to about 40% w/w of total composition, and said isopropyl myristate is present at a concentration of from about 5 to about 15% w/w of total composition.

12. The foamable composition of claim 6, wherein said fatty acid ester comprises one or more of sorbitan monolaurate; sorbitan mono palmitate; sorbitan monooleate, sorbitan trioleale; sorbitan sesquioleate; sorbitan isostearate; sorbitan sesquistearate; sucrose monopalmitate; sucrose monostearate; sucrose distearate; sucrose polystearate; propylene glycol stearate; glyceryl monooleate; glyceryl monostearate; glyceryl myristate, or combinations thereof.

13. The foamable composition of claim 12, wherein said fatty acid ester comprises glyceryl monooleate at a concentration in the range of from about 0.1% to about 7% w/w of total composition.

14. The foamable composition of claim 13, wherein said surfactant comprises sucrose polystearate.

15. The foamable composition of claim 14, wherein said sucrose polystearate is present at a concentration of up to about 6% w/w of total composition.

16. The foamable composition of claim 15, wherein said surfactant further comprises sucrose distearate.

17. The foamable composition of claim 16, wherein said sucrose distearate is present at a concentration of up to about 4% w/w of total composition.

18. The foamable composition of claim 17, further comprising a co-emulsifier.

19. The foamable composition of claim 18, wherein said co-emulsifier comprises one or more of beeswax, lanolin, cetyl alcohol, stearyl alcohol, cetostearyl alcohol, stearic acid and palmitic acid.

20. The foamable composition of claim 19, wherein said beeswax is present at a concentration of up to about 6% w/w of total composition.

21. The foamable composition of claim 19, wherein said lanolin is present at a concentration of up to about 7% w/w of total composition.

22. The foamable composition of claim 21, further comprising panthenol.

23. The foamable composition of claim 22, wherein said panthenol is present at a concentration of up to about 5% w/w of total composition.

24. The foamable composition of claim 23, further comprising aloe vera extract.

25. The foamable composition of claim 24, wherein said aloe vera extract is present at a concentration of up to about 1% w/w of total composition.

26. The foamable composition of claim 25, further comprising a preservative which is not benzalkonium chloride.

27. The foamable composition of claim 26, wherein said preservative is selected from the group consisting of sodium benzoate, benzoic acid, benzyl alcohol, a paraben or salt thereof, imidurea, potassium sorbate, sorbic acid, phenoxyethanol, chlorocresol, or bronopol, and mixtures thereof.

28. The foamable composition of claim 27, further comprising a self emulsifying surfactant selected from the group consisting of a mixture of glyceryl stearate with PEG-100 stearate, a mixture of sorbitan stearate with sucrose cocoate, PEG-30 dipolyhydroxystearate, ceteth-2, and mixtures thereof.

29. The foamable composition of claim 28, wherein said mixture of glyceryl stearate with PEG-100 stearate is present at a concentration of up to about 1% w/w of total composition, and said mixture of sorbitan stearate with sucrose cocoate is present at a concentration of up to about 1% w/w of total composition, said PEG-30 dipolyhydroxystearate is present at a concentration of up to about 1% w/w of total composition, and said ceteth-2 is present at a concentration of up to about 5% w/w of total composition.

30. The foamable composition of claim 29, further comprising at least one of bisabolol, a chelating agent, a hydrophilic solvent, and an emulsion stabilizer.

31. The foamable composition of claim 30, wherein said bisabolol is present at a concentration of up to about 1% w/w of the total composition.

32. The foamable composition of claim 30, wherein said chelating agent comprises EDTA or a sodium or calcium salt thereof.

33. The foamable composition of claim 30, wherein said chelating agent comprises disodium EDTA at a concentration of up to about 1% w/w total composition.

34. The foamable composition of claim 30, wherein said hydrophilic solvent is selected from the group consisting of propylene glycol and glycerin.

35. The foamable composition of claim 34, wherein said propylene glycol is present at a concentration of from about 1% to about 3% w/w total composition.

36. The foamable composition of claim 30, wherein said emulsion stabilizer comprises magnesium sulfate.

37. The foamable composition of claim 36, wherein said magnesium sulfate is present at a concentration of up to about 1% w/w of total composition.

38. The foamable composition of claim 26, wherein said paraben is selected from the group consisting of methyl paraben, methylethylparaben, propyl paraben, butylparaben, salts thereof and mixtures thereof.

39. The foamable composition of claim 38, wherein said paraben is selected from the group consisting of methyl paraben, methyl paraben sodium, propyl paraben, propyl paraben sodium, or mixtures thereof.

40. The foamable composition of claim 39, wherein said methyl paraben or said methyl paraben sodium is present at a concentration of up to about 0.2% w/w of total composition.

41. The foamable composition of claim 40, wherein said propyl paraben or said propyl paraben sodium is present at a concentration of up to about 0.1% w/w of total composition.

42. The foamable composition of claim 41, adapted for delivery from a pressurized container, wherein a foam is formed upon expulsion from said container.

43. Use of the foamable composition of claim 42, for topical application.

44. The foamable composition of claim 42, for cosmetic application.

45. The foamable composition of claim 42, for use in treating or preventing a skin condition.

46. The foamable composition of claim 45, wherein said skin condition comprises one or more of diaper rash, psoriasis, eczema, urticaria, scabies, acne, alopecia areata, bullous pemphigoid, dermatitis, atopic dermatitis, impetigo, keloids, pruritis, rosacea, vitiligo, burns, irritation, inflammation, fungal infection, dry skin, skin allergies or diabetic skin conditions.

47. The foamable composition of claim 42, for providing a skin soothing and/or anti-itching effect.

48. The foamable composition of claim 47, for use on an incontinent human adult.

49. The foamable composition of claim 48, wherein said human adult is selected from the group consisting of a geriatric patient, a physically handicapped patient and a mentally handicapped patient.

50. The foamable composition of claim 42, for use in a patient prone to develop a skin condition.

51. The foamable composition of claim 50, wherein said patient is selected from a group consisting of athletes, soldiers and obese people.

52. The foamable composition of claim 42, further comprising one or more propellant gases.

53. The foamable composition of claim 2, comprising at least one of zinc oxide, hyaluronic acid, zinc hyaluronate, or a plant extract.
